(19) United States
(12) Patent Application Publication
(10) Pub. No.: US 2016/0151296 A1
(43) Pub. Date: Jun. 2, 2016

(54) LONG DELAYED RELEASE LAXATIVE

(71) Applicant: EMPIRE TECHNOLOGY DEVELOPMENT LLC, Wilmington, DE (US)

(72) Inventors: Benjamin Matthew AUSTIN, Bangor (AU); Michael Keoni MANION, Cronulla (AU); George Charles PEPPOU, Hornsby Heights (AU)

(73) Assignee: Empire Technology Development LLC, Wilmington, DE (US)

(21) Appl. No.: 14/892,753

(22) PCT Filed: May 20, 2014
(86) PCT No.: PCT/US2014/038792

§ 371 Date: Nov. 20, 2015

Related U.S. Application Data
(60) Provisional application No. 61/826,117, filed on May 22, 2013.

(51) Int. Cl. A61K 9/50

(52) U.S. Cl. CPC A61K 9/5052; A61K 9/5036

(57) ABSTRACT

Disclosed herein are compositions and methods for treating or preventing the build-up or entrapment of fecal matter in the colon. The compositions and methods disclose a delayed release laxative that provides for the release of the laxative if colon-specific conditions are achieved, otherwise the laxative passes out of the body. The condition specific release of laxatives prevents harmful side effects from excessive use of non-delayed release laxatives.

### CROSS-REFERENCE TO RELATED APPLICATIONS

This application claims priority to U.S. Application No. 61/826,117 filed May 22, 2013, the entire contents of which is hereby incorporated by reference in its entirety.

### BACKGROUND

The following description is provided to assist the understanding of the reader. None of the information provided or references cited is admitted to be prior art to the present technology.

Constipation refers to bowel movements that are infrequent or hard to pass. Constipation is a common cause of painful defecation. Severe constipation includes obstipation (failure to pass stools or gas) and fecal impaction.

Diverticulitis occurs when diverticula, pouches in the wall of the colon, are formed due to increased pressure pushing on the walls of the colon. If the pouches get inflamed or infected, it is called diverticulitis. Diverticulitis can be very painful.

The collection of fecal matter in diverticula can lead to the formation of compacted and calcified faecoliths. Faecoliths increase the incidence of diverticulitis by damaging the endothelium in the diverticula leading to bleeding and infection.

Bowel obstruction is a mechanical or functional obstruction of the intestines, preventing the normal transit of the products of digestion. It can occur at any level distal to the duodenum of the small intestine and is a medical emergency. The condition is often treated conservatively over a period of 2-5 days with the patient's progress regularly monitored by an assigned physician.

### SUMMARY

Provided herein are compositions and methods for the treatment and prevention of constipation, the formation of faecoliths, diverticulitis, and related disorders or conditions. In particular, compositions comprising long delay release laxatives are provided, as are methods of making and using the same.

In one aspect, the disclosure provides a composition comprising a long delay release laxative. In some embodiments, the composition includes a coating encapsulating the laxative. In some embodiments, the coating is configured to prevent laxative release for at least 2 days after administration. In some embodiments, the coating is configured to prevent laxative release for at least about 3 days, 4 days, 5 days, 6 days or at least about 7 days after administration.

In some embodiments, the coating includes one or more compounds, which can be degraded under colon specific conditions. In some embodiments, the coating includes a compound which can be degraded by colonic bacteria. In some embodiments, the coating includes a pH sensitive compound. In some embodiments, the pH sensitive compound is degraded under colon specific conditions. In some embodiments, the colon specific conditions include a pH between about 7-8.

In some embodiments, the coating includes one or more of the following compounds: azopolymers, azohydrogels, chitosan, pectins, guar gum, dextrans, inulin, lactulose, amylose, cyclodextrins, alginates, locust bean gum, chondroitin sulphate, cellulose, boswellia gum, fructan, sugar alcohols (e.g. maltitol, sorbitol, xylitol and isomalt), and ethyl cellulose. Additionally or alternatively, in some embodiments, the coating includes polymerized lactose and/or cross-linked fibrin.

In some embodiments, the coating includes polymerized lactose and cross-linked fibrin.

In some embodiments, the coating is 50-200% by weight of the core material weight.

In some embodiments, the laxative includes a hyperosmotic agent. For example, in some embodiments, the laxative includes one or more of the following compounds: macrogol (polyethylene glycol), saline (various solutions are used: sodium phosphate, potassium sodium tartarate, magnesium citrate, magnesium hydroxide, magnesium sulfate) and sorbitol. Additionally or alternatively, in some embodiments, the laxative includes one or more stimulant or irritant agents. Exemplary stimulant or irritants include one or more of cascara, buckthorn, senna extract, aloin, phenolphthalein and bisacodyl.

In some embodiments, the laxative includes a softening agent. In some embodiments, the laxative is in the form of a liquid.

In some embodiments, the laxative composition is in the form of microspheres.

In some embodiments, the laxative composition is in the form of a liquid suspension of microspheres, wherein the microspheres include a coating encapsulating the laxative.

In some embodiments, the composition comprises one or more active agents encapsulated with the laxative. For example, in some embodiments, the composition includes one or more of the following active agents: a lubricant, an antibiotic, anti-inflammatory, agents capable of decalcification of the faecolith, and agents which increase the rate of release of laxative from surrounding particles.

In another aspect, the disclosure provides a method for treating, preventing ameliorating the symptoms of diverticulitis, the method comprising: administering to a subject a composition comprising a laxative encapsulated by a coating, wherein the coating prevents release of the laxative for at least for at least 2 days after administration. Additionally or alternatively, in some embodiments, the coating is configured to prevent laxative release for at least about 3 days, 4 days, 5 days, 6 days or at least about 7 days after administration.

In another aspect, the disclosure provides a method for treating or preventing the formation of compacted faecoliths, the method comprising: administering to a subject a composition comprising a laxative encapsulated by a coating, wherein the coating prevents release of the laxative for at least 2 days after administration. Additionally or alternatively, in some embodiments, the coating is configured to prevent laxative release for at least about 3 days, 4 days, 5 days, 6 days or at least about 7 days after administration.

In another aspect, the disclosure provides a method for treating or preventing bowel obstruction, the method comprising: administering to a subject a composition comprising a laxative encapsulated by a coating, wherein the coating prevents release of the laxative for at least 2 days after administration. Additionally or alternatively, in some embodiments, the coating is configured to prevent laxative release for at least about 3 days, 4 days, 5 days, 6 days or at least about 7 days after administration.

In another aspect, the disclosure provides a method for treating or preventing constipation, the method comprising: administering to a subject a composition comprising a laxative encapsulated by a coating, wherein the coating prevents release of the laxative for at least at least 2 days after administration. Additionally or alternatively, in some embodiments, the coating is configured to prevent laxative release for at least about 3 days, 4 days, 5 days, 6 days or at least about 7 days after administration.

In some embodiments of the methods, the coating includes one or more compounds which are degraded by colonic bacteria. Additionally or alternatively, in some embodiments of the methods, the coating includes one or more compounds which are degraded under colon-specific conditions. Additionally or alternatively, in some embodiments of the methods, the colon-specific condition includes a pH of between about 7-8.

In some embodiments of the methods, the coating includes one or more of the following compounds: azopolymers, azohydrogels, chitosan, pectins, guar gum, dextrans, inulin, lactulose, amylose, cyclodextrins, alginates, locust bean gum, chondroitin sulphate, cellulose, boswellia gum, fructan, sugar alcohols (e.g. maltitol, sorbitol, xylitol and isomalt), and ethyl cellulose.

Additionally or alternatively, in some embodiments of the methods, the coating includes one or more of the following compounds: polymerized lactose and cross-linked fibrin.

In some embodiments of the methods, the coating has a thickness 50-200% of the core material weight.

In some embodiments of the methods, the laxative includes a hyperosmotic agent.

In some embodiments of the methods, the hyperosmotic agent includes one or more hyperosmotic agents. In some embodiments, the one or more hyperosmotic agents include: macrogol (polyethylene glycol), saline (various solutions are used: sodium phosphate, potassium sodium tartarate, magnesium citrate, magnesium hydroxide, magnesium sulfate) and sorbitol.

In some embodiments of the methods, the laxative further includes a softening agent.

In some embodiments of the above methods, the laxative is in the form of a liquid.

In some embodiments of the methods, the laxative is in the form of microspheres.

In some embodiments of the above methods, the composition is in the form of a liquid suspension of microspheres, wherein the microspheres includes the coating encapsulating the laxative.

In some embodiments of the methods, one or more active agents are encapsulated with the laxative.

In some embodiments of the methods, one or more active agents includes one or more of a lubricant, an antibiotic, anti-inflammatory, and agents capable of decalcification of the faecolith, agents which increase rate of release of laxative from surrounding particles.

The foregoing summary is illustrative only and is not intended to be in any way limiting. In addition to the illustrative aspects, embodiments and features described above, further aspects, embodiments and features will become apparent by reference to the following drawings and the detailed description.

### BRIEF DESCRIPTION OF THE DRAWINGS

FIG. 1 is an illustrative overview of a system in which the encapsulated laxative is activated after 5 days due to fecal entrapment in diverticulum.

FIG. 2 is a diagram illustrative of the delayed release mechanism after 5 days within a forming faecolith.

### DETAILED DESCRIPTION

In the following detailed description, reference is made to the accompanying drawings, which form a part hereof In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Disclosed herein are compositions and methods for treating and preventing constipation, the formation of faecoliths, diverticulitis, and related disorders or conditions. The compositions and methods disclosed herein comprise long-delayed release laxatives. In some embodiments, a laxative, and optionally one or more additional active agents (e.g., an antibiotic, anti-inflammatory agent, etc.), is encapsulated with a coating configured to prevent laxative release for at least 2-7 days or more after administration to a subject (long delay release). For example, in some embodiments, the long-delay release laxative may be administered orally, e.g., in the form of a capsule or tablet. Once in the subject's gastrointestinal system, the laxative capsule will eventually come into contact with fecal matter. If the fecal matter is expelled from the body within e.g., 2 days of laxative administration, the laxative is excreted from the body (e.g., with a bowel movement). However, if the fecal matter remains in the body for 2 days or more, the coating begins to break down and laxative is released.

Accordingly, the disclosed composition and methods help reduce the side effects of prolonged use of laxatives, as the laxatives are released when needed (e.g., only after a prolonged period in the subject's gastrointestinal tract).

The technology is described herein using several definitions, as set forth throughout the specification.

For the purposes of this disclosure and unless otherwise specified, “a” or “an” means “one or more.”

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the term “laxative” refers to any foods, compounds or drugs taken to loosen, break down, lubricate, or soften stool, or aid in the expulsion of stool or any combination thereof Additionally, “laxative” also includes any combination of a laxative and another agents or compounds which facilitate, ameliorate, treat, or prevent fecal build-up or fecal entrapment in the colon. The following described laxatives and supplemental agents or compounds are exemplary, non-limiting, and could be used in combination. It is understood that the laxative agent may optionally be combined with one or more additional active agents in the methods and compositions disclosed herein.

In some embodiments, the laxative may include a hyperosmotic agent. Hyperosmotic agents draw water into the stool from the surrounding tissue to prevent the stool from hardening. Hyperosmotic agents are fast acting but typically are not used as a long term treatment strategy as they may irritate the colon and can cause dehydration and electrolyte imbalances. Common, non-limiting examples include Movicol®, lactulose, solutions of polyethylene glycol, electrolytes (e.g. sodium chloride, sodium bicarbonate, potassium chloride, and sometimes sodium sulfate), macrogol (polyethylene glycol), saline (various solutions are used: sodium phosphate, potassium sodium tartarate, magnesium citrate, magnesium hydroxide, magnesium sulfate) and sorbitol. In some embodiments, the laxative may include lubricating agents. Lubricating agents are typically oil based and keep the stool soft, encouraging movement through the colon. A common, non-limiting example is mineral oil.

As used herein, the term “stimulant/irritant agents” refer to agents that act on the intestinal mucosa or nerve plexus, altering water and electrolyte secretion, which increase the activity of the colon to move stool out. In some embodiments, the laxative may include one or more stimulant/irritant agents. Exemplary, non-limiting examples include cascara, buckthorn, senna extract, aloin, phenolphthalein, cholera toxin, and bisacodyl.

As used herein, the term “active agent” refers to any agent, compound, therapeutic agent, or pharmaceutical compound that would aid in the treatment, prevention, or have a prophylactic effect, or a therapeutic effect for symptoms related to fecal build-up and/or entrapment in the colon. In some embodiments, the active agent may be encapsulated with the laxative. In some embodiments, the active agent may be provided separately from the laxative, and, for example, may be administered before, simultaneous to, or after laxative administration. In some embodiments, active agents include, but are not limited to, one or more of an antibiotic, decalcifying agent, demineralizing agent, an anti-inflammatory agent, an analgesic, and a softening agent, such as docusate.

As used herein the term “release agent” refers to any compound that facilitates the release of a laxative and any other active agents from a coating, e.g., a coating encapsulating the laxative. For example, if a laxative coating includes amylose, amylase, could be encapsulated within the microsphere with the laxative as a release agent. The release and activation of amylase would facilitate the degradation of surrounding microspheres.

As used herein, “long delay” or “long release,” when used in reference to a laxative containing compound of the present disclosure means that in some embodiments, the coating is configured to prevent laxative release for at least about 2 days, 3 days, 4 days, 5 days, 6 days or at least about 7 days after administration.

As used herein “coating,” with reference to coating encapsulating the laxative, refers to any compound, natural or synthesized, that coats or encapsulates the laxative to contribute to a long delay of the release of the laxative. In some embodiments, microsphere encapsulation of laxative may be used to encapsulate the laxative and to help facilitate uniform distribution of laxative throughout digested materials. Exemplary, non-limiting coatings may include microbially degradable coatings, such as polysaccharide (or similar), azopolymers, azohydrogels, chitosan, pectins, guar gum, dextrans, inulin, lactulose, amylose, cyclodextrins, alginates, locust bean gum, chondroitin sulphate, cellulose, boswellia gum, fructan, sugar alcohols (e.g. maltitol, sorbitol, xylitol and isomalt), and ethyl cellulose.

As used herein, the term “microbially degraded” or “microbially degradable” refers to a compound (e.g., useful as a coating in the present disclosure) that is selectively degraded by the microflora typically found in the colon.

As used herein, “colon specific conditions” refers to conditions that mimic the colonic environment. One example of a colon-specific condition is having a pH of between about 7-8, and/or the presence of colonic microflora.

As used herein, the terms “treating” or “treatment” or “alleviation” refers to both therapeutic treatment and prophylactic or preventative measures, wherein the object is to prevent or slow down (lessen) the targeted pathologic condition or disorder. For example, a subject is successfully “treated” for diverticulitis if, after receiving a therapeutic amount of the encapsulate laxative according to the methods described herein, the subject shows observable and/or measurable reduction fecal packed diverticula or the subject maintain normal bowel movements without excessive laxative use side effects. It is also to be appreciated that the various modes of treatment or prevention of medical conditions as described are intended to mean “substantial,” which includes total but also less than total treatment or prevention, and wherein some biologically or medically relevant result is achieved.

As used herein, “prevention” or “preventing” of a disorder or condition refers to a compound that reduces the occurrence of the disorder or condition in the treated sample relative to an untreated control sample, or delays the onset or reduces the severity of one or more symptoms of the disorder or condition relative to the untreated control sample.

### Laxative Encapsulation for Long Delay Release

Disclosed herein are compositions comprising long delay release laxatives. In some embodiments, the laxative is encapsulated with a coating configured to prevent or delay laxative release for at least 2 days after administration to a subject.

The long delayed release laxative compositions disclosed herein are not limited by the type of laxative used, or the form of the laxative. By way of example, but not by way of limitation, laxatives may be hyperosmotic agents or lubricating agents. Common hyperosmotic agents include, but are not limited to, Movicol®, lactulose, solutions of polyethylene glycol, electrolytes (e.g. sodium chloride, sodium bicarbonate, potassium chloride, and sometimes sodium sulfate), macrogol (polyethylene glycol), saline (various solutions are used: sodium phosphate, potassium sodium tartarate, magnesium citrate, magnesium hydroxide, magnesium sulfate) and sorbitol. A common example of a lubricating agent includes, but is not limited to, mineral oil. Laxatives may be in solid, liquid or gel form, or may be formed into microspheres or nanospheres for encapsulation. In some embodiments, the nanospheres may have an average diameter of about 100 nm to 1000 nm, 200 nm to 900 nm, 300 nm to 800 nm, 400 nm to 700 nm, 500 nm to 600 nm. Additionally or alternatively, in some embodiments, the microspheres may have an average diameter of about 1 μm to 20 μm, 20 μm to 50 μm, 50 μm to 100 μm, 100 μm to 200 μm, 200 μm to 300 μm, 300 μm to 400 μm, 400 μm to 500 μm, 500 μm to 600 μm, or 600 μm to 700 μm.

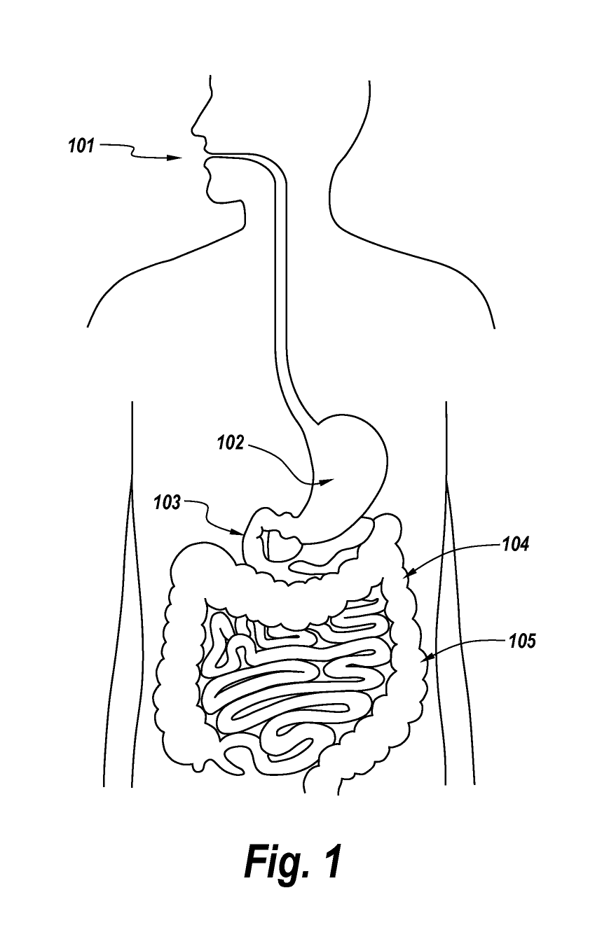

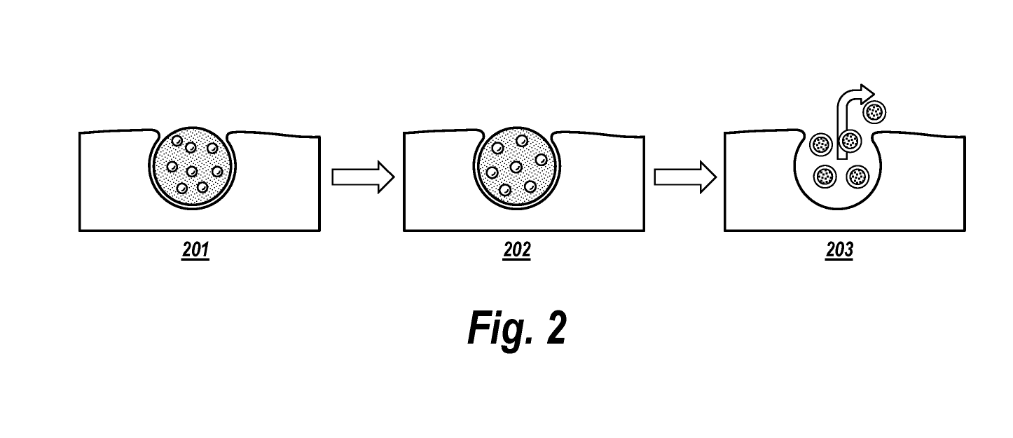

In some embodiments, the laxative compositions disclosed herein may be coated or encapsulated to ensure long delayed release (e.g., at least about 2-7 days or longer after administration). FIGS. 1 and 2 present illustrative embodiments of the disclosed technology. The figures are provided to aid the reader in understanding the disclosed compositions and methods and are not intended to be limiting.

With reference to FIG. 1, after taking a long delay release laxative e.g., in tablet form, orally 101, the tablet breaks down in the stomach into individual microspheres 102 that would spread throughout the gastrointestinal (“GI”) tract 103. In some embodiments, the surface coating of the microsphere may be prepared such that it is affected (e.g., broken down) by the presence of the microbes found in the large intestine. Under normal condition, e.g., where passage of fecal matter is less than two days, no laxative is released 104. The laxative microspheres pass through the system and may be excreted (e.g., with a bowel movement). However, if the microsphere encapsulated laxative is trapped (e.g. in diverticule), and remains in the GI tract for greater than 2 days, the coating begins to break down in the presence of the colonic microflora, releasing the laxative 105.

With reference to FIG. 2, when fecal matter with the laxative microspheres are trapped in diverticula 201, the coating of the microspheres will slowly degrade. Colonic conditions such as pH and/or colonic bacteria effect the rate of degradation. If the fecal matter stays trapped for an extended period, e.g., 5 days in this particular example 202, then the coating will completely degrade and release the laxative into the surrounding area and break-up the fecal matter, which becomes part of the normal bowel movement 203. However, if the fecal matter passes before the 5 days, in this particular example, then the laxative is never released and passes out of the body.

Hence, by achieving the desired thickness and composition of the encapsulation, the time delay for microsphere erosion and laxative release can be controlled. Thus, not only is the laxative released at a time that is effective for the subject, it is also be released at a location that is effective for the subject, e.g., at a particular site of need (e.g., in formed fecoliths in diverticula).

One method to achieve the desired delayed release is the use of a microbially degradable coating around the laxative. These coatings are selectively degraded by the microflora found in the colon and not typically found in other regions of the GI tract. In some embodiments, the degradation time of the coating may be designed to be longer than the normal bowel passage time. In some embodiments the coating may be one or more of the following compounds: polysaccharide (or similar), azopolymers, azohydrogels, chitosan, pectins, guar gum, dextrans, inulin, lactulose, amylose, cyclodextrins, alginates, locust bean gum, chondroitin sulphate, cellulose, boswellia gum, fructan, sugar alcohols (e.g. maltitol, sorbitol, xylitol and isomalt), and ethyl cellulose.

By way of example, but not by way of limitation, an example of a combination of encapsulation compounds is the COLAL™ system(Alizyme, Cambridge, UK). The COLAL™ system uses a film of amylose and ethyl cellulose to encapsulate a medication. The release time of the medication is controlled by varying the thickness of the film and the ratio of amylose to ethyl cellulose.

In one exemplary embodiment, the laxative may first be coated with an amylose coating. Coating thickness could then be increased by adding additional layers of amylose, increasing coating thickness, and/or then modifying the polysaccharide composition to increase the delay in release.

In some embodiments, coating or encapsulation thickness may be measured by percent weight of core material (e.g., the laxative). For example, a 200% by core weight coating indicates that 1 g of core material includes 2 g of coating compounds. In one embodiment, the coating is about 50-200% of the core weight material. In another embodiment, the coating is about 75-175% of the core weight material. In another embodiment, the coating is about 100-150% of the core weight material.

In some embodiments, delay in degradation of the coating may be achieved by the use of pH sensitive coating, such as amylose-ethyl cellulose systems, for example the CODES™ systems (Yamanouchi Pharmaceutical Co., Ltd., Japan). pH sensitive coating, such as enteric coatings, may be used to help the laxative bypass the stomach with little or no degradation and to help the laxative target the colon. Enteric coatings include, but are not limited to, cellulose acetate 1,2 benzedicarboxylate, hexadecan-1-01, cellulose ethyl ether, liquid glucose, cellulose 2-hydroxyethylether, cellulose 2-hydroxypropylether, cellulose 2-hydroxypropyl methyl ether, cellulose hydrogen 1,2 benzene dicarboxylate 2-hydroxypropylmethyl ether, maltodextrin, cellulose methyl ether, polymethacrylates, shellac, confectioner's sugar, titanium oxide, carnauba wax, microcrystalline wax, zein, gelatin, polyvinyl ethanol, sodium alginate and stearic acid, methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers.

Additionally or alternatively, cross-linking polymers can be used to extend release time. Examples of cross-linking polymers include, but are not limited to, chitosan, fibrin, glyoxal, glutaraldehyde, epichlorohydrin, 1-ethyl-3-(3-dimethylaminopropyl) carbodimide together with N-hydroxysuccinimide, thrombin, and genipin.

Additionally or alternatively, any of the above-described methods may be combined to achieve the desired time delay release of the laxative.

### Degradation of the Coating in the Colon

Colonic bacteria include microorganisms that live in the digestive tracts of animals. The microorganisms perform a host of useful functions, such as fermenting unused energy substrates, training the immune system, preventing growth of harmful, pathogenic bacteria, regulating the development of the gut, producing vitamins for the host (such as biotin and vitamin K), and producing hormones to direct the host to store fats. In some embodiments, the coating of the laxative may be designed to be degraded by colonic bacteria, or under colon-specific conditions, using any of the coatings described above.

As noted previously, colon specific conditions refers to an environment which mimics the colon. In some embodiments, colon-specific conditions may be sufficient to trigger the release of laxative from its encapsulation. One example of a colon-specific condition is having a pH of between about 5-10. Another example of a colon-specific condition is having a pH of between about 6-9. Yet another example of a colon-specific condition is having a pH of between about 7-8.

To prevent release of the laxative in the stomach, enteric coatings can be used to protect the laxative from degradation in the highly acidic stomach. However, the colon has a more neutral pH, which can be used to trigger the degradation of the coating to release the laxative. Another example of a colon specific condition is targeting specific colonic bacteria to degrade the coating. During diverticulitis, some colonic bacteria are more prevalent. Coatings can be targeted to be degraded by colonic bacteria that are elevated during episodes of diverticulitis. Yet, another example of a colon-specific condition is having the disease or condition that needs to be treated, for example, but not limited to: having constipation, having calcified faecoliths, formation of diverticula, having diverticulitis, having fecal compaction, obstipation, bowel obstruction, or any combination thereof. In some embodiments, if the colon-specific conditions are not met, then the laxatives are not released.

Additionally or alternatively, in some embodiments it may also be useful for the coating encapsulated laxative to accumulate in the diverticula or other target areas. For example, a specific coating may be formulated to aid in the adhesion of the encapsulated laxative to trapped fecal matter in the diverticula.

Additionally or alternatively, in some embodiments, a laxative composition may be formulated with a double (or multiple) encapsulation, such as to protect an outer coating from an encapsulated release agent. For example, if a laxative coating includes amylose, amylase could be encapsulated in a smaller sphere within the microsphere to facilitate degradation of other microspheres, as described above. Multiple encapsulation would lead to controlled release of the compounds in the microsphere.

### Conditions Treated or Prevented

The present technology relates to the treatment or prevention of the build-up or entrapment of fecal matter in the colon. When fecal matter builds up or is trapped in the colon, it can become compacted or calcified into faecoliths. Compaction and faecoliths are hard and sometime painful to pass. Chronic compaction or formation of faecoliths can damage the endothelium of the colon and lead to bleeding, inflammation, and infection.

Example of conditions or disease that could be treated with the present technology include, but are not limited to: constipation, obstipation, fecal compaction, calcified faecoliths, bowel obstruction, and diverticulitis. Constipation refer to bowel movements that are infrequent or hard to pass. Obstipation is the failure to pass stools or gas. Fecal compaction is solid, immobile bulk of feces that develop due to constipation. Diverticulitis occurs when feces gets trapped in diverticula, pouches in the colon, and the feces calcifies into faecoliths, which can cause damage to the colon endothelium.

The mammal treated in accordance present methods can be any mammal, including, for example, farm animals, such as sheep, pigs, cows, and horses; pet animals, such as dogs and cats; laboratory animals, such as rats, mice and rabbits. In some embodiments, the mammal is a human.

### Active Agents or Release Agents

The laxative can be formulated with additional active agents. In some embodiments, the laxative may be co-encapsulated with an antibiotic and/or an anti-inflammatory. In some embodiments, this can provide further prophylactic or therapeutic treatment targeted to the area where infection or inflammation may occur. Additionally or alternatively, in some embodiments, the laxative may be encapsulate with therapeutic agents that aid in the decalcification or demineralization of faecolith. Additionally or alternatively, in some embodiments, the laxative may be encapsulate with a softening agent to aid in passage of the fecal matter.

In some embodiments, the laxative may be encapsulated with release agents that result in a feed-forward mechanism upon release, to result in a rapid release profile from all trapped microspheres. For example, amylose coated microspheres may have encapsulated amylase which becomes active when released, to aid in the cleavage of nearby microspheres.

### Modes of Administration

For the purpose of oral therapeutic administration, the laxative may be provided in the form of a tablet, capsule, liquid, gelatin capsule, paste, powder, or combination thereof. In some embodiments, the oral therapeutic may be an encapsulated liquid suspension of microspheres. Additionally or alternatively, in some embodiments, microspheres of the laxative compound may be included into functional foods or beverages as well as tablet or capsule form. In another embodiment, the laxative could be delivered as a suppository.

In one embodiment, the timing of therapeutic administration is once daily. In some embodiments, the timing of therapeutic administration is twice daily. In some embodiments, the timing of therapeutic administration is every other day. In some embodiments, the timing of therapeutic administration is once every 5 days. In some embodiments, the timing of therapeutic administration is at least once week. In some embodiments, the dosage is between about 1 g to 50 g, 5 g to 45 g, 10 g to 40 g, 15 g to 35 g, or about 20 g to 30 g of the laxative. In some embodiments, a dosage includes about 1 g, 2 g, 3 g, 4 g, 5 g, 6 g, 7 g, 8 g, 9 g, 10 g, 11 g, 12 g, 15 g, 20 g, 25 g, 30 g, 35 g, 40 g, 45 g, 50 g, 55 g, 60 g or more of the laxative.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

### Equivalents

The embodiments, illustratively described herein, may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent compositions, apparatuses, and methods within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like, include the number recited and refer to ranges which can be subsequently broken down into sub-ranges as discussed above. Finally, as will be understood by one skilled in the art, a range includes each individual member.

While certain embodiments have been illustrated and described, it should be understood that changes and modifications can be made therein in accordance with ordinary skill in the art without departing from the technology in its broader aspects as defined in the following claims.

1. A composition comprising:
a laxative;
a coating encapsulating the laxative,
wherein the coating is configured to prevent laxative release for at least 2 days after administration to a subject.

2. The composition of claim 1, wherein the coating is configured to prevent laxative release for at least about 3 days, 4 days, 5 days, 6 days or at least about 7 days after administration.

3.-5. (canceled)

6. The composition of claim 1, wherein the coating comprises one or more biodegradable compounds selected from the group consisting of:
azopolymers, azohydrogels, chitosan, pectins, guar gum, dextrans, inulin, lactulose, amylose, cyclodextrins, alginates, locust bean gum, chondroitin sulphate, cellulose, boswellia gum, fructan, maltitol, sorbitol, xylitol, isomalt, and ethyl cellulose.

7. The composition of claim 1, wherein the coating further comprises one or more compounds selected from the group consisting of polymerized lactose and cross-linked fibrin.

8. The composition of claim 1, wherein the coating further comprises one or more enteric compounds selected from the group consisting of: cellulose acetate 1,2 benzedicarboxylate, hexadecan-1-01, cellulose ethyl ether, liquid glucose, cellulose 2-hydroxyethylether, cellulose 2-hydroxypropylether, cellulose 2-hydroxypropyl methyl ether, cellulose hydrogen 1,2 benzene dicarboxylate 2-hydroxypropylmethyl ether, maltodextrin, cellulose methyl ether, polymethacrylates, shellac, confectioner's sugar, titanium oxide, carnauba wax, microcrystalline wax, zein, gelatin, polyvinyl ethanol, sodium alginate and stearic acid, methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers and cellulose carboxymethyl ether sodium salt.

9. The composition of claim 1, wherein the coating is 50-200% by weight of the core material weight.

10. The composition of claim 1, wherein the laxative comprises one or more hyperosmotic agents, wherein the one or more hyperosmotic agents are selected from the group consisting of: macrogol, saline, and sorbitol.

11. (canceled)

12. The composition of claim 10, wherein the laxative comprises one or more stimulant/irritant agents selected from the group consisting of cascara, buckthorn, senna extract, aloin, phenolphthalein and bisacodyl.

13. The composition of claim 1, wherein the laxative further comprises a softening agent.

14.-16. (canceled)

17. The composition of claim 1, further comprising one or more active agents encapsulated with the laxative, wherein the one or more active agents is selected from the group consisting of an antibiotic, an anti-inflammatory, a decalcifying agent, a demineralizing agent, analgesic, and a coating release agent.

18. (canceled)

19. A method for treating, preventing, and/or ameliorating the symptoms of diverticulitis, the method comprising:
administering to a subject a composition comprising a laxative encapsulated by a coating, wherein the coating prevents release of the laxative for at least 2 days after administration to a subject.

20. The method of claim 19, wherein the coating is configured to prevent laxative release for at least about 3 days, 4 days, 5 days, 6 days or at least about 7 days after administration.

21. A method for treating or preventing the formation of compacted faecoliths, the method comprising:
administering to a subject a composition comprising a laxative encapsulated by a coating, wherein the coating prevents release of the laxative for at least 2 days after administration to a subject.

22.-24. (canceled)

25. A method for treating or preventing constipation, the method comprising:
administering to a subject a composition comprising a laxative encapsulated by a coating, wherein the coating prevents release of the laxative for at least 2 days after administration to a subject.

26.-29. (canceled)

30. The method of claim 19, wherein the coating comprises one or more biodegradable compounds selected from the group consisting of azopolymers, azohydrogels, chitosan, pectins, guar gum, dextrans, inulin, lactulose, amylose, cyclodextrins, alginates, locust bean gum, chondroitin sulphate, cellulose, boswellia gum, fructan, maltitol, sorbitol, xylitol, isomalt, and ethyl cellulose.

31. (canceled)

32. (canceled)

33. The method of claim 19, wherein the coating has a thickness 50-200% of the core material weight.

34. The method of claim 19, wherein the laxative comprises one or more hyperosmotic agents, wherein the one or more hyperosmotic agents are selected from the group consisting of: macrogol, saline, and sorbitol.

35. (canceled)

36. The method of claim 19, wherein the laxative further comprises a softening agent.

37.-39. (canceled)

40. The method of claim 19, further comprising one or more active agents encapsulated with the laxative, wherein the one or more active agents is selected from the group consisting of an antibiotic, an anti-inflammatory, a decalcifying agent, a demineralizing agent, analgesic, and a coating release agent.

41. (canceled)
